(19) United States
(12) Patent Application Publication
(10) Pub. No.: US 2004/0072825 A1
(43) Pub. Date: Apr. 15, 2004

(54) 2-(2-(4-((2r)-2-methyl-3-(10h-phenothiazin-10-yl)propyl)-1-piperazinyl)-ethoxy) ethanol, process for the preparation thereof, pharmaceutical compositions containing said compound and therapeutic uses thereof

(76) Inventors: Yves Lamberty, Braine-le-Chateau (BE); Jacques Timmermans, Ittre (BE); Tony Waegemans, Leuven (BE); Guy Bodson, Tintigny (BE); Febienne Broeders, Bruxelles (BE)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/433,832

(22) PCT Filed: Nov. 15, 2001
(86) PCT No.: PCT/EP01/13227

(30) Foreign Application Priority Data
Dec. 19, 2000 (EP) 00127741.7

(51) Int. Cl. A61K 31/5415; C07D 417/04

(52) U.S. Cl. 514/225.8; 544/44

(57) ABSTRACT

The present invention relates to the compound 2-(2-(4-((2R)-2-methyl-3-(10H-phenothiazin-10-yl)propyl)-1-piperazinyl)ethoxy)ethanol, and its pharmaceutically acceptable salts, as well as to processes for the preparation thereof. It also relates to pharmaceutical compositions containing the said compounds, as well as to therapeutic uses thereof.

The present invention relates to the novel compound 2-(2-(4-((2R)-2-methyl-3-(10H-phenothiazin-10-yl)propyl)-1-piperazinyl)ethoxy)ethanol, and its pharmaceutically acceptable salts, as well as to processes for the preparation thereof. It also relates to pharmaceutical compositions containing the said compounds, as well as to uses thereof.

British patent n^{o} 861,420 describes the compound 2-(2-(4-(2-methyl-3-(10H-phenothiazin-10-yl)propyl)-1-piperazinyl)ethoxy)ethanol, with the following formula Dixyrazine

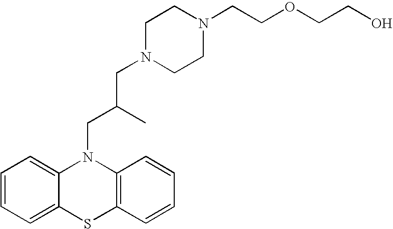

This compound is known under the international non-proprietary name (INN) dixyrazine. A salt of the compound, dixyrazine dihydrochloride, has a melting point 192° C. Dixyrazine can be used for therapeutic purposes, for example as an antipsychotic agent. It is also known as a neuroleptic agent and sold under the trademark ESUCOS.

It is further known that dixyrazine possesses antiemetic properties (Acta Anaesthesiol. Scand., 1999, 43(2), 191-195; and Acta Oncologica, 1997, 36(2), 229-230). Analgesia with morphine is often associated with postoperative nausea and vomiting, and it is mentioned that prophylactic administration of dixyrazine reduces the incidence and severity of vomiting.

It has now surprisingly been found that the previously undisclosed (R) enantiomer of dixyrazine differs in a completely unpredictable manner from the (S) enantiomer, by having not only potent prophylactic activity against nausea and vomiting but lacking pro-emetic properties surprisingly found in the (S) enantiomer.

As a result of this unexpected property the (R) enantiomer of dixrazine, and its pharmaceutically acceptable salts, are suitable for the treatment and prevention of emesis. The compound is particularly useful for the treatment of emesis in cancerotherapy.

Accordingly, the present invention relates to the (R) enantiomer of dixyrazine which has the R absolute configuration, and its pharmaceutically acceptable salts. The said compounds are substantially free from the (S) enantiomer, which has the S absolute configuration. The term “substantially free from the (S) enantiomer” as used herein refers to a stereochemical mixture of dixyrazine containing at least 90% by weight of (R) enantiomer and 10% or less by weight of the (S) enantiomer. In a more preferred embodiment the term means that the dixyrazine mixture contains at least 98% by weight of the (R) enantiomer, and 2% or less of the (S) enantiomer. In a most preferred embodiment the term means that the mixture contains greater than 99% by weight of the (R) enantiomer and includes pure (R) enantiomer.

The term “pharmaceutically acceptable salt” as used herein means addition salts with pharmaceutically acceptable non-toxic organic and inorganic acids, such as acetic, benzoic, oleic, undecylenic, salicylic, ascorbic, glycolic, lactic, malic, gluconic, tartaric, citric, succinic, malonic, fumaric, maleic, pamoic, methane sulfonic, ethane sulfonic, benzene sulfonic, p-toluene sulfonic, p-chloro-benzene sulfonic, saccharinic, lauryl sulphonic, hydrochloric, hydrobromic, sulphuric, phosphoric, propanoic, pyruvic and the like. The present invention preferably concern 2-(2-{4-[(2R)-2-methyl-3-(10H-phenothiazin-10-yl)propyl]-1-piperazinyl}ethoxy)ethanol and its dihydrochloride salt.

The present invention also concerns processes for the preparation of the (R) enantiomer of dixyrazine and its pharmaceutically acceptable salts.

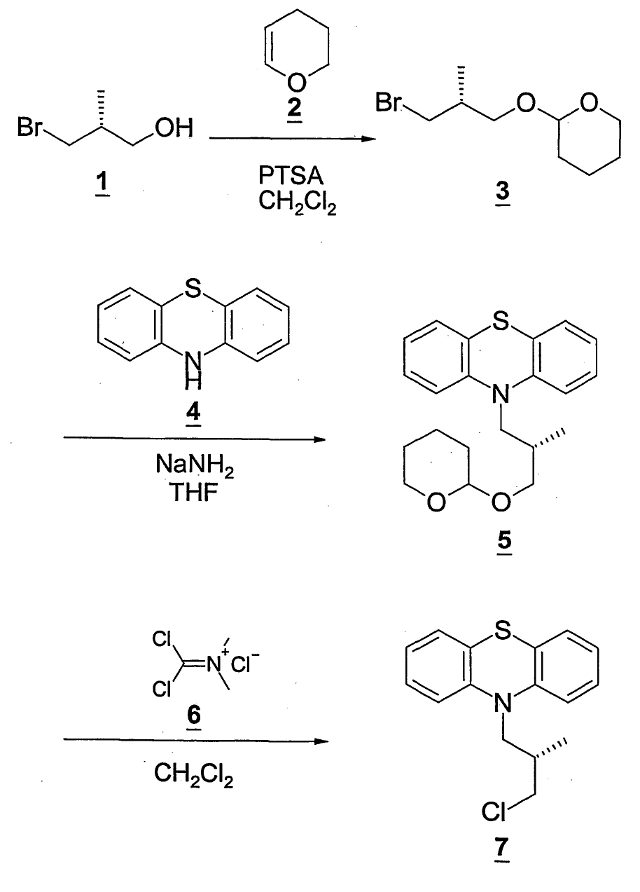

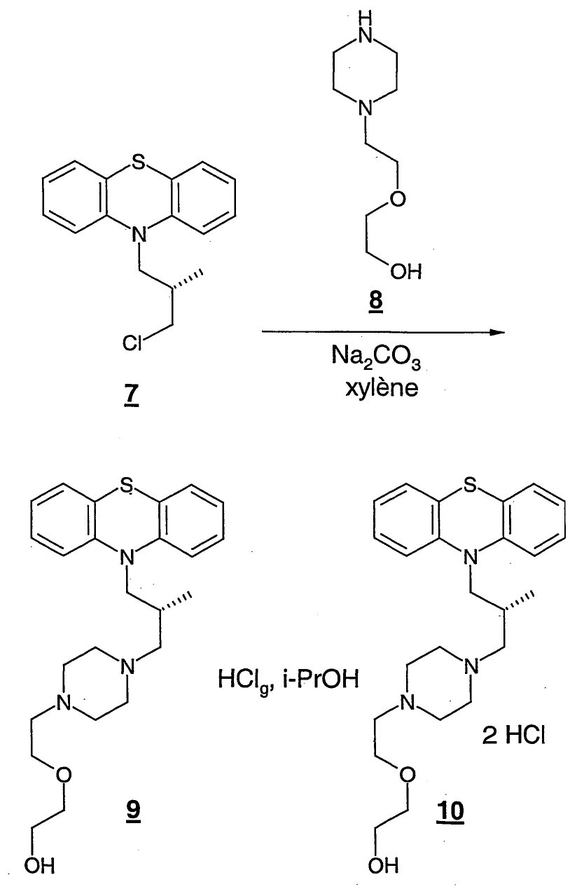

Such a process is illustrated by the reaction scheme of FIG. 1.

A process of preparation includes the following steps:

2-(2-{4-[(2R)-2-methyl-3-(10H-phenothiazin-10-yl)propyl]-1-piperazinyl}ethoxy)ethanol may be obtained by reaction of 10-[(2S)-3-chloro-2-methylpropyl]-10H-phenothiazine with 2-[2-(1-piperazinyl)ethoxy]ethanol. This reaction is carried out in the presence of an acid acceptor, such as an alkali metal carbonate, and optionally in the presence of a small amount of alkali metal iodide, in an inert organic solvent, for example xylene, preferably at a temperature in the region of the reflux temperature. The obtained free base may be converted to its dihydrochloride salt according to conventional methods known to the person skilled in the art. As an example, free base is dissolved in an inert organic solvent, such as isopropanol, and a solution of gaseous hydrochloric acid in an alcohol, such as methanol, is added. 2-(2-{4-[(2R)-2-methyl-3-(10H-phenothiazin-10-yl)propyl]-1-piperazinyl}ethoxy)ethanol dihydrochloride is obtained in a form of a white powder.

10-[(2S)-3-chloro-2-methylpropyl]-10H-phenothiazine may be obtained by reaction of 10-[(2S)-2-methyl-3-(tetrahydro-2H-pyran-2-yloxy)propyl]-10H-phenothiazine with (dichloromethylene)dimethylammonium chloride (Vilsmeier reagent). This reaction may be carried out in an inert organic solvent, for example dichloromethane, between −20 and 0° C.

10-[(2S)-2-methyl-3-(tetrahydro-2H-pyran-2-yloxy)propyl]-10H-phenothiazine may be obtained by reaction of 10H-phenothiazine with 2-{[(2R)-3-bromo-2-methylpropyl]oxy}tetrahydro-2H-pyran. This reaction is carried out in the presence of sodium amide, in an inert organic solvent, for example tetrahydrofuran, between 40 and 60° C.

2-{[(2R)-3-bromo-2-methylpropyl]oxy}tetrahydro-2H-pyran may be obtained by reaction of (2R)-3-bromo-2-methyl-1-propanol with 3,4-dihydro-2H-pyran. This reaction is carried out in the presence of para-toluenesulphonic acid monohydrate (PTSA), in an inert organic solvent, for example dichloromethane, between −10 and 5° C.

The individual enantiomers may also be prepared in conventional manner for example by chromatographic separation of a racemic mixture of final or intermediate compounds.

The present invention concerns also 2-(2-{4-[(2R)-2-methyl-3-(10H-phenothiazin-10-yl)propyl]-1-piperazinyl}ethoxy)ethanol and its pharmaceutically acceptable salts for use as a medicament.

The present invention concerns also 2-(2-{4-[(2R)-2-methyl-3-(10H-phenothiazin-10-yl)propyl]-1-piperazinyl}ethoxy)ethanol and its pharmaceutically acceptable salts for use as an antiemetic agent.

The present invention also concerns a pharmaceutical composition containing the (R) enantiomer of dixyrazine or a pharmaceutically acceptable salt thereof free of the (S) enantiomer and further containing a suitable carrier.

A pharmaceutical composition is considered to be free of the (S) enantiomer if at least 95 weight-% of the active substance is represented by the (R) enantiomer and in preferred embodiments at least 98 weight-% of the active substance is represented by the (R) enantiomer.

The pharmaceutical composition of the invention can reduce, ameliorate or eliminate one or more symptoms of at least one type of emesis.

The compounds of the present invention can be administered either orally in the form of solid or liquid compositions, in the form of tablets, pills, dragees, gelatine capsules, solutions or syrups, or parenterally in the form of injectable solutions or suspensions. Pharmaceutical forms such as solutions or tablets are prepared according to conventional pharmaceutical methods. The compounds of the invention may be mixed with a solid or liquid non-toxic pharmaceutically acceptable carrier and optionally with a dispersant, a stabilizer and where necessary, colorants and sweeteners. Solid pharmaceutical excipients for the preparation of tablets or capsules include starch, talc, calcium carbonate, lactose, sucrose and magnesium stearate. As an example, a tablet contains 10 mg of the compound of the present invention, 25 mg of lactose, 4 mg of cellulose, 1.5 mg of a product sold under the trademark Povidone, 1.5 mg of magnesium stearate. As an example, oral drop contains 2.2 g of the compound of the present invention, 200 mg of a flavouring agent and a vehicle such as propyleneglycol.

For the preparation of parenterally injectable solutions or suspensions, the compound of the invention may be solubilized or suspended in an aqueous or a nonaqueous vehicle, optionally with a buffer system and where necessary an isotonicity adjusting agent and a preservative.

A non-limiting example of such a composition for intravenous injection contains 20 mg of the product of the invention, anhydrous sodium sulfite, sodium citrate, with pH adjusted to 4.5 with hydrochloric acid and injectable water added to make up the solution to 2 ml.

The percentage of active compound in the pharmaceutical compositions can vary within wide limits depending upon the mode of administration and the condition of the patient.

The present invention further concerns a therapeutic use of the (R) enantiomer of dixyrazine and of its pharmaceutically acceptable salts.

The (R) enantiomer of dixyrazine and its pharmaceutically acceptable salts are useful in the treatment or prevention of emesis. In particular they are useful in the treatment or prevention of emesis in chemotherapy-induced nausea and vomiting.

Furthermore, they also are useful in the treatment or prevention of emesis as premedication for post-operative nausea and vomiting.

The term “emesis” refers to the process commonly known as vomiting or retching, wherein the stomach is evacuated through the oesophagus and mouth due to strong muscular contractions in the abdomen. Emesis is usually accompanied by nausea and feelings of strong malaise and discomfort.

The present invention provides a method for preventing or treating, in humans and mammals, disorders or conditions associated with nausea or vomiting, such as radiation induced emesis, cancerotherapy, HIV-therapy, motion sickness, migraine, post operative nausea, Ménière's disease and related disorders, which comprises the administration of an effective amount of the (R) enantiomer of dixyrazine or a pharmaceutically acceptable salt thereof.

Control of emesis is crucial in providing optimal cancer care and to avoid delay in, or refusal of, chemotherapy. Uncontrolled emesis can cause depression, anxiety, dehydration, malnutrition, clinically relevant weight loss, and general interference with quality of daily life.

Uncontrolled or uncontrollable emesis is the single most important reason for rejection of chemotherapic cancer care. Moreover, it may reduce the potential to reach optimal therapeutic doses.

The (R) enantiomer of dixyrazine and its pharmaceutically acceptable salts are useful for the manufacture of a medicament for preventing or treating disorders or conditions associated with nausea or vomiting.

In particular, the (R) enantiomer of dixyrazine and its pharmaceutically acceptable salts are effective antiemetic agents, useful as an adjunctive therapy in cancer treatment to alleviate nausea and vomiting induced by chemo- or radio-therapeutics.

The present invention relates to a method of reducing emesis as a side-effect associated with administering or delivering chemotherapy, radiotherapy or immunosuppresive therapy to a patient comprising administering the (R) enantiomer of dixyrazine or a pharmaceutically acceptable salt thereof to said patient.

The (R) enantiomer of dixyrazine and its pharmaceutically acceptable salts thereof are also effective to treat or prevent emesis associated with surgical, anaesthesial or psychological stress, certain disease states (such as migraine, headaches), radiotherapy, chemotherapy, radiation poisoning and toxic substances.

The pharmaceutical composition of the present invention does not present the side effects due to the (S) enantiomer.

According to another embodiment, the present invention concerns also the following chemical compounds:

10-[(2S)-2-methyl-3-(tetrahydro-2H-pyran-2-yloxy)propyl]-10H-phenothiazine;

10-[(2S)-3-chloro-2-methylpropyl]-10H-phenothiazine;

10-[(2R)-2-methyl-3-(tetrahydro-2H-pyran-2-yloxy)propyl]-10H-phenothiazine;

10-[(2R)-3-chloro-2-methylpropyl]-10H-phenothiazine; and

10-[(2)-2-methyl-3-(tetrahydro-2H-pyran-2-yloxy)propyl]-10H-phenothiazine.

These chemical compounds can be used as synthesis intermediates.

The present invention concerns also the (S) enantiomer of dixyrazine and its pharmaceutically acceptable salts thereof.

A process for preparing the chemical compounds is described above.

The following non-limiting examples are provided for the purpose of illustration only.

### EXAMPLES

### Example 1

2-{[(2R)-3-bromo-2-methylpropyl]oxy}tetrahydro-2H-pyran (compound 3) is prepared as follows.

90 ml of methylene chloride are introduced into a 250 ml three-necked flask equipped with a magnetic stirrer and a thermometer and are cooled to 0° C. 6 g (0.039 mol) of (2R)-3-bromo-2-methyl-1-propanol (compound 1) and 72 mg (0.39 mmol) of para-toluenesulphonic acid monohydrate (PTSA), followed dropwise by 10.68 ml (0.195 mol) of 3,4-dihydro-2H-pyran (compound 2), are subsequently added. The stirred reaction mixture is maintained at a temperature slightly below 0° C. for 30 minutes. The reaction mixture is washed with a saturated aqueous sodium hydrogencarbonate solution, decanted, and the aqueous phase is extracted with methylene chloride. The combined organic phases are dried over anhydrous sodium sulphate, filtered and concentrated to dryness under reduced pressure.

15.8 g of crude 2-{[(2R)-3-bromo-2-methylpropyl]oxy}tetrahydro-2H-pyran (compound 3) are thus isolated in the form of an oil still containing a certain amount of starting 3,4-dihydro-2H-pyran (compound 2).

10-[(2S)-2-methyl-3-(tetrahydro-2H-pyran-2-yloxy)propyl]-10H-phenothiazine (compound 5) is prepared as follows.

80 ml of tetrahydrofuran and 4.74 g (0.0238 mol) of 10H-phenothiazine (compound 4) are introduced, under a nitrogen atmosphere, into a 250 ml three-necked flask equipped with a reflux condenser, a dropping funnel and a magnetic stirrer. This solution is brought to 50° C. with stirring and under a nitrogen atmosphere. 4.65 g (0.1193 mol) of sodium amide are then added thereto portionwise. After 30 minutes, 15.8 g of 2-{[(2R)-3-bromo-2-methylpropyl]oxy}tetrahydro-2H-pyran (compound 3), isolated in the form of a crude oil and dissolved in 40 ml of tetrahydrofuran, are added dropwise to this reaction mixture. After the end of the addition, the solution is stirred for a further 30 minutes at 50° C. and then cooled. Water is added and the mixture is extracted twice with methylene chloride. The combined organic phases are washed with a saturated aqueous sodium chloride solution and then dried over anhydrous sodium sulphate, filtered and concentrated to dryness under reduced pressure.

17.78 g of crude 10-[(2S)-2-methyl-3-(tetrahydro-2H-pyran-2-yloxy)propyl]-10H-phenothiazine (compound 5) are thus isolated in the form of an oil still comprising a certain amount of 10H-phenothiazine (compound 4).

10-[(2S)-3-chloro-2-methylpropyl]-10H-phenothiazine (compound 7) is prepared as follows.

80 ml of methylene chloride and the 17.78 g of 10-[(2S)-2-methyl-3-(tetrahydro-2H-pyran-2-yloxy)propyl]-10H-phenothiazine (compound 5) obtained in the form of a crude oil from the previous step are introduced into a 250 ml three-necked flask, maintained under a nitrogen atmosphere, equipped with a reflux condenser, a thermometer and a magnetic stirrer. This stirred solution cooled to −10° C. 8.7 g (0.054 mol) of (dichloromethylene)dimethylammonium chloride (compound 6: Vilsmeier reagent) are then added thereto portionwise. The temperature of the stirred reaction mixture is maintained between −10° C. and 0° C. After 5 hours, a further 1 g of (dichloromethylene)dimethylammonium chloride (compound 6) is added to the reaction mixture, which is stirred for a further 1.5 hours at between −10° C. and 0° C. 30 ml of water are subsequently added thereto, the mixture is decanted and the aqueous phase is extracted three times with methylene chloride. The combined organic phases are concentrated to dryness under reduced pressure.

26.54 g of crude 10-[(2S)-3-chloro-2-methylpropyl]-10H-phenothiazine (compound 7) are thus obtained. This compound is purified by chromatography on silica gel. The useful fractions are combined and concentrated to dryness under reduced pressure.

8.43 g of 10-[(2S)-3-chloro-2-methylpropyl]-10H-phenothiazine (compound 7) are obtained in the form of an oil.

2-(2-{4-[(2R)-2-methyl-3-(10H-phenothiazin-10-yl)propyl]-1-piperazinyl}ethoxy)ethanol (compound 9) is prepared as follows.

5 g of 10-[(2S)-3-chloro-2-methylpropyl]-10H-phenothiazine (compound 7), 3 g of 2-[2-(1-peperazinyl)ethoxy]ethanol (compound 8), 10 g of sodium carbonate and 10 ml of xylene are introduced, under a nitrogen atmosphere, into a 100 ml three-necked flask equipped with a thermometer, a magnetic stirrer and a condenser of the Dean and Stark type. The suspension obtained is brought to 150° C., with stirring and under a nitrogen atmosphere, for 30 h. The reaction mixture is subsequently brought back to room temperature with stirring. 10 ml of water are added thereto. The mixture is decanted and the organic phase is washed twice with water. The combined aqueous phases are extracted twice with toluene. The combined organic phases are dried over anhydrous sodium sulphate, filtered and concentrated to dryness under reduced pressure.

4.36 g of crude 2-(2-{4-[(2R)-2-methyl-3-(10H-phenothiazin-10-yl)propyl]-1-piperazinyl}ethoxy)ethanol (compound 9) are obtained in the form of an oil.

Purification of 2-(2-{4-[(2R)-2-methyl-3-(10H-phenothiazin-10-yl)propyl]-1-piperazinyl}ethoxy)ethanol (compound 9) is made as follows.

Crude oil obtained above (4.36 g) is combined with 1.48 g of same product obtained in another assay, then purified by chromatography to give 3.5 g of 2-(2-{4-[(2R)-2-methyl-3-(10H-phenothiazin-10-yl)propyl]-1-piperazinyl}ethoxy)ethanol (compound 9). These 3.5 g of compound 9 are combined with 6.46 g of compound 9 obtained in a comparable fashion, chromatographed on silica gel. 8.7 g of 2-(2-{4-[(2R)-2-methyl-3-(10H-phenothiazin-10-yl)propyl]-1-piperazinyl}ethoxy)ethanol (compound 9) are thus isolated with an HPLC purity of 96.4%.

2-(2-{4-[(2R)-2-methyl-3-(10H-phenothiazin-10-yl)propyl]-1-piperazinyl}ethoxy)ethanol dihydrochloride (compound 10) is prepared as follows.

The 8.7 g of 2-(2-{4-[(2R)-2-methyl-3-(10H-phenothiazin-10-yl)propyl]-1-piperazinyl}ethoxy)ethanol (compound 9) free base are dissolved in 27 ml of isopropanol at room temperature with stirring. 29 ml of a 1.4 molar solution of gaseous hydrochloric acid in methanol are added thereto. The thick suspension which forms is diluted with 18 ml of isopropanol. After stirring for two hours at room temperature, the crystals formed are filtered off, washed on the filter and dried to constant weight under reduced pressure.

6 g of 2-(2-{4-[(2R)-2-methyl-3-(10H-phenothiazin-10-yl)propyl]-1-piperazinyl}ethoxy)ethanol dihydrochloride (compound 10) are obtained in a form of a white powder having the following analytical characteristics:

Analysis for C24H33N3O2S 2HCl in %

| | | | | |
|---|---|---|---|---|
| C: | calculated: | 57.59 | found: | 57.77 |
| H: | calculated: | 7.05 | found: | 7.00 |
| N: | calculated: | 8.40 | found: | 8.37 |
| Cl: | calculated: | 14.17 | found: | 13.93 |
| M.p.: | 246.6° C. (DSC) | | | |
| Enantiomeric excess | 100% | | | |
| (chiral HPLC): | | | | |

Chromatographic separations are performed on silicagel 60 Merck, particle size 15-40 μm, reference 1.15111.9025.

M.p. represents melting point in ° C. They are determined by the onset temperature on a Perkin Elmer DSC 7.

Enantiomeric excesses were determined by chiral chromatography using Chiralpak AD as chiral stationary phase (220 nm, 15° C., Eluent: propanol 10%, isohexane 90%+DEA 0.1%).

### Example 2

2-(2-{4-[(2S)-2-Methyl-3-(10H-phenothiazin-10-yl)propyl]-1-piperazinyl}ethoxy)ethanol dihydrochloride is synthesized in the same way starting from (2S)-3-bromo-2-methyl-1-propanol. Its analytical characteristics are as follows:

Analysis for C24H33N3O2S. 2HCl in %

| | | | | |
|---|---|---|---|---|
| C: | calculated: | 57.59 | found: | 57.47 |
| H: | calculated: | 7.05 | found: | 7.11 |
| N: | calculated: | 8.40 | found: | 8.35 |
| Cl: | calculated: | 14.17 | found: | 13.93 |
| M.p.: | 250.2° C. (DSC) | | | |
| Enantiomeric excess | 100% | | | |
| (chiral HPLC): | | | | |

### Example 3: Pharmacological Tests

Cisplatin-induced emesis in ferrets is largely considered as the most suitable animal model for the study of drug activity against chemotherapy-induced vomiting in man (Georges L. King. Animal models in the study of vomiting. Can. J. Physiol. Pharmacol. 68, 260-268, 1988).

Cisplatin (cis-diamine dichloroplatinum) is a chemotherapeutic drug which has a powerful emetic effect. Cisplatin is one of the early anticancer drugs, notoriously known for its bad tolerance. The test is a very hard test.

Cisplatin-induced emesis in ferrets results in very severe emesis. Drugs active in this model could therefore be considered as very potent against emesis.

Three compounds, dixyrazine dihydrochloride and its (R) and (S) enantiomers were tested for their activity against vomiting induced by cisplatin in ferrets following single oral administration.

The study involved 14 groups of 8 male ferrets weighing between 0.93 kg and 1.45 kg purchased from the company MARSHALL FARMS (NEW YORK, U.S.). Groups were defined as follows:

group 1: control group dosed with the vehicle of test substances (sterile water),

group 2: method-control group dosed with a method-control substance, granisetron (MDL 72222, RBI®), at a dose of 0.3 mg/kg,

group 3 to 6: group dosed with diyyrazine dihydrochloride at 4 increasing doses of 7.5, 15, 30 and 60 mg/kg,

group 7 to 10: group dosed with the (R) enantiomer of dixyrazine dihydrochloride at 4 increasing doses of 7.5, 15, 30 and 60 mg/kg,

group 11 to 14: group dosed with the (S) enantiomer of drazine dihydrochloride at 4 increasing doses of 7.5, 15, 30 and 60 mg/kg.

The product Granisetron is a specific 5-HT3 serotonin receptor antagonist which is currently used in humans to treat chemotherapy-induced vomiting (F. Mitchelson. Pharmacological agents affecting emesis, a review (part I). Drugs, 43 :295-315, 1992).

On the day prior to the test, animals were fasted and allowed access only to water.

On the morning of the day of the study, free access to food was allowed for 30 minutes. Subsequently, animals were again put on to a water-only fast for 30 minutes before administration of the three compounds, the vehicle or the method-control substance which were administrated orally in a volume of 5 ml/kg.

Cisplatin was administrated by the intravenous route at a dose of 115 mg/m² and in a volume of 115 ml/m² (i.e. approximately at 10 mg/kg), 60 minutes after treatment. Animals were observed for 5 hours following the administration of cisplatin. Emesis was characterised by abdominal contractions, which were associated with expulsion or attempted expulsion of stomach contents.

The time to onset of emesis was noted. The number of episodes of vomiting and the number of abdominal retches associated with episodes of emesis were also noted.

Under the experimental conditions adopted, the racemic compound and the (R) enantiomer induced an antiemetic effect at the highest dose of 60 mg/kg. This effect was characterised by a statistically significant suppression of vomiting in 5 and 4 animals out of 8 respectively.

Moreover for the (R) enantiomer, a tendency to increase the time to onset of emesis was seen. For the racemic compound, no effect was seen on this parameter. The (S) enantiomer had no antiemetic effect whatever the dose tested. On the contrary at the highest dose of 60 mg/kg, a statistically significant decrease in the time of onset of emesis was noted suggesting a proemetic effect of the (S) enantiomer.

Under the same conditions, the method-control substance (Granisetron, 0.3 mg/kg, p.o. (per os)) administered 1 hour before intravenous administration of cisplatin induced, as expected, a significant antiemetic effect. This effect was characterised by a suppression of vomiting induced by cisplatin in 5 animals out of 8 and an increase in the time to onset of emesis in the 3 remaining animals, showing the validity of the method used.

The results are summarized in table 1.

The obtained results show that dixyrazine dihydrochloride and the (R) enantiomer of dixyrazine dihydrochloride significantly reduce cisplatin-induced emesis at a dose of 60 mg/kg p.o.; and that the (S) enantiomer of dixyrazine dihydrochloride is inactive in preventing cisplatin-induced emesis and can even worsens the effect of cisplatin.

The (R) enantiomer of dixyrazine dihydrochloride also shows a clear tendency to delay the latency to onset of the first vomiting episode at the highest dose, whereas the racemic compound did not alter this variable. Thus, the (R) enantiomer of dixyrazine dihydrochloride appears to present an advantage over the racemate. It is plausible that the lack of activity of the racemate in reducing the onset of emesis is due to the negative effect of the (S) enantiomer on this parameter. Therefore, the (R) enantiomer of dixyrazine dihydrochloride should reveal a better activity in humans compared to the racemic compound, the anti-emetic activity of which is not optimal due to the presence of the (S) enantiomer pro-emetic effect.

The (S) enantiomer of dixyrazine dihydrochloride appears to possess undesirable pro-emetic properties.

TABLE 1

| | | Proportion of | |
|---|---|---|---|
| | Dose | animal | Time to onset of |
| Compounds | (mg/kg p.o.) | vomiting | emesis (minutes) |
| Vehicle | | 8/8 | 77 |
| Granisetron | 0.3 | 3/8* | 160* |
| Racemic | 7.5 | 8/8 | 90 |
| Racemic | 15 | 5/8 | 76 |
| Racemic | 30 | 7/8 | 73 |
| Racemic | 60 | 3/8* | 81 |
| (R) | 7.5 | 5/8 | 81 |
| (R) | 15 | 8/8 | 80 |
| (R) | 30 | 5/8 | 81 |
| (R) | 60 | 4/8* | 134 |
| (S) | 7.5 | 8/8 | 87 |
| (S) | 15 | 8/8 | 87 |
| (S) | 30 | 7/8 | 82 |
| (S) | 60 | 7/8 | 68* |

*represents a significant difference (P < 0.05) compared with vehicle treated animals.
Racemic represents dixyrazine dihydrochloride (racemate).
(R) represents the (R) enantiomer of dixyrazine dihydrochloride.
(S) represents the (S) enantiomer of dixyrazine dihydrochloride.
Proportion of animal vomiting represents the number of animals vomiting in comparison of the total number of animals included in the study.

### Example 4

This study reports the selectivity and affinity of dixrazine arid its enantiomers for some receptors.

With respect to H1 histaminic receptor, and D2, D3 and D4.4 dopaminergic receptors relevant for anti-emetic activity (Synapse, 24(3), 1996, 224-232; Eur. J. Pharmacol. 272, 1995, 21-30; Drugs 43(3), 1992, 295-315 and 443-463), as well as with regard to H1 histaminic receptors and serotonergic 5-HT2 receptors relevant for tranquilizing/anxrolytic and sleep improving effect (biomed&Pharmacother., 54, 2000,263-267; Psychopharmacology 142, 1999, 318-326), the affinity of dixyrazine is stereoselective, with the (R) enantiomer being the more potent with pIC50 ranging from 6.8 to 9.7.

The test substances, biological materials and experimental protocols used in the study are described in Moguilevsky N. et al. (1994) Eur. J. Pharmacol. 224:489-495; List S. J. (1981) Proc. Natl. Acad. Sci.78:2620-2624; MacKenzie R. G. et al. (1994) Eur. J. Pharmacol. 266:79-85; Van Tol et al., (1992) Nature, 358:149-152.

The rat striatum membranes (D2 receptor), the rat cerebral cortex (5-HT2 receptor) and the CHO cell culture (human H1, human D3 and D4.4 receptors) are incubated with the test drug and the radioligand, filtered (GF/B or GF/C, Whatman or Packard; Filtermat A, Walac), then washed with cold buffer using a filtration unit ‘Cell Harvester’ (Brandel, Packard or Tomtec). The bound radioactivity is measured using a scintillation liquid (Formula 989 or Microscint 0, Packard) or solid (Meltilex B/HS, Wallac), and a scintillation counter (LS 6000, Beckman; Topcount, Packard; Betaplate, Wallac).

The test drug is added to the binding assay at two concentrations, 0.1 μM and 10 μM. Each experiment was performed in duplicate (n=2). In parallel, a positive control (concentration of reference substance causing 50% inhibition of radioligand specific binding) is tested to validate the assay.

For competition experiments, membranes are incubated in the buffer containing the radioligand and increasing concentrations of unlabeled test substances. Each binding assay is validated with a reference compound tested in parallel.

The results with respect to H1, D2, D3, D4.4 and 5-HT2 receptors are given as a percentage of inhibition of the radioligand specific binding:

% inhibition=100−[((BI-NSB)/(BO-NSB))×100]

where BO and BI represent the total binding in the absence and presence of the unlabelled drug (in dpm/assay) and NSB is the amount of non specific binding (in dpm/assay).

Analysis of raw data and pIC50 calculation is performed by computerized non-linear curve fitting methods using a set of equations describing competitive interactions between labelled and unlabeled ligands which obey the law of mass action.

Dixyrazine dihydrochloride, (R) enantiomer free base, (R) enantiomer dihydrochloride, (S) enantiomer free base and (S) enantiomer dihydrochloride were tested.

The results are summarized in table 2 and indicate that the binding of specific radloligands to D2, D3, D4.4 dopaminergic receptors, 5-HT 2 serotonergic receptors and H1 histaminic receptors is inhibited more than 90% in the presence of 10 μM dixyrazine.

Experiments performed with the (R) and (S) enantiomers of dixyrazine (free base) show that they are stereoselective in their binding to these targets with the (R) enantiomer being more potent than the (S) enantiomer.

In a second set of experiments, the pIC50 of dixyrazine and the (R) and (S) enantiomers dihydrochloride are evaluated at the H1 receptor, D2, D3 and D4.4, and 5-HT2 receptors.

The results in table 3 indicate that the affinity of dixyrazine and its eantiomers is stereoselective with the (R) enantiomer being the more potent at D2, D3, D4.4 dopaminergic receptors, 5-HT2 serotonergic receptors and H1 histaminic receptors being relevant for anti-emetic activity.

The stereoselectivity ratio between the two enantiomers is presented in table 4. The stereoselectivity ratio is calculated as follows: IC50 value of (S) enantiomer/IC50 value of (R) enantiomer. The ratio is calculated on the basis of pIC50.

This example demonstrates that the (R) enantiomer of dixyrazine shows the best affinities towards H1 and selected dopaminergic receptors. It is particularly suited to treat nausea and vomiting and avoid polypharmacy. In effect, Van Liessum et al. (Van Liessum PA, De Milder PHM and De Haan LD. Nausea and vomiting induced by cytostatic agents. Scand. J. Gastroenterol, 1989, 24 (suppl.171), 106-111) have proposed to use a combination of antiemetics as the appropriate way to block dopaminergic, cholinergic and histaminergic transmission, which are instrumental in vomiting. The (R) enantiomer of dixyrazine with its high affinities for histaminic H1 receptors and 3 important dopaminergic receptors is therefore particularly suited to fulfill this role, but as a single chemical entity.

Another advantage of the (R) enantiomer of dixyrazine over the current treatment by 5-HT3 antagonists is that, due to its affinity for H1 and D2, it will alleviate the sensation of nausea. Interviews of patients in cancerotherapy have revealed that ondansetron (5-HT3 antagonist) for example, is clearly effective in preventing vomiting but has no clear-cut effect against the nausea (personal interview of patients in cancerotherapy).

Anticipatory nausea and vomiting in cancerotherapy can be defined as the occurrence of nausea and vomiting before the administration of cytostatics in patients who have vomited during previous cycles of chemotherapy (Van Liessum et al., 1989). This syndrome is associated with anxiety and agitation. Relaxation or hypnosis may improve the syndrome. The advantage of the (R) enantiomer of dixyrazine over 5-HT3 antagonists is that it possesses tranquilizing, anxiolytic and sleep improving effects due to its antihistaminic H1 activity and to its antiserotonergic 5-HT2 activity and can therefore be expected to calm the patient (to alleviate anticipatory anxiety), prevent the syndrome, improve the outcome of nausea and emesis during chemotherapy and consequently provide patients with a better quality of life during chemotherapy (Support Care Cancer, 2001, 9, 366-371; biomed & Pharmacother., 54, 2000,263-267; Psychopharmacology 142, 1999, 318-326; Anticancer Res., 2000, 20, 4777-4784)

TABLE 2
Effect of dixyrazine and enantiomers on the specific radioligand binding to
the receptors studied.

| | | | (S) enantiomer | | (R) enantiomer | |
|---|---|---|---|---|---|---|
| | Dixyrazine | | (free base) | | (free base) | |
| Receptors | 0.1 μM | 10 μM | 0.2 μM | 10 μM | 0.1 μM | 10 μM |
| D4.4 | 24 | 104 | — | 27 | 40 | 99 |
| D3 | 84 | 100 | — | 74 | 81 | 100 |
| D2 | | 94 | | 89 | | 100 |
| H1 | | 100 | | 101 | | 101 |
| 5-HT3 | — | 26 | | | | |
| 5-HT2 | | 102 | | 101 | | 102 |

The results are expressed as a percent inhibition of control specific binding. The compounds are tested in duplicate; the results are expressed as mean values. Symbol-represents an inhibition<10%.

TABLE 3
Affinity pIC50 (-log IC50) for selected receptors

| | | (R) enantiomer | (S) enantiomer |
|---|---|---|---|
| Receptor | Dixyrazine | dihydrochloride | dihydrochloride |
| D3 | 7.3 | 7.3 | 5.5 |
| D2 | 8.3 | 8.6 | 6.1 |
| D4.4 | 6.4 | 6.8 | 4.6 |
| H1 | 9.3 | 9.7 | 8.3 |
| 5-HT2 | 8.2 | 8.4 | 7.2 |

IC50 represents the concentration of the test substance inhibiting 50% of the specific binding of the radioligand. It is determined in competitive binding experiments by measuring the binding of a single concentration of a radioactive ligand at equilibrium with various concentrations of the unlabeled test substance. pIC50=−log IC50

TABLE 4
Stereoselectivity ratio for (R) enantiomer dihydrochloride

| Receptor | Stereoselectivity ratio |
|---|---|
| D3 | 63 |
| D2 | 316 |
| D4.4 | 158 |
| H1 | 25 |
| 5-HT2 | 16 |

1. Process for preparing 2-(2-(4-((2R)-2-methyl-3-(10H-phenothiazin-10-yl)propyl)-1-piperazinyl)ethoxy)ethanol characterized in that it comprises a reaction of 10-[(2S)-3-chloro-2-methylpropyl]-10H-phenothiazine with 2-[2-(1-piperazinyl)ethoxy]-ethanol.

2. Pharmaceutical composition containing 2-(2-(4-((2R)-2-methyl-3-(10H-phenothiazin-10-yl)1-piperazinyl)ethoxy)ethanol or its pharmaceutically acceptable salts free of the (S) enantiomer and further containing a suitable carrier.

3. 2-(2-(4-((2R)-2-methyl-3-(10H-phenothiazin-10-yl)propyl)-1-piperazinyl)ethoxy)ethanol, or its pharmaceutically acceptable salts for use as a medicament.

4. 2-(2-(4-((2R)-2-methyl-3-(10H-phenothiazin-10-yl)propyl)-1-piperazinyl)ethoxy)ethanol, or its pharmaceutically acceptable salts for use as an antiemetic agent.

5. Therapeutic use of 2-(2-(4-((2R)-2-methyl-3-(10H-phenothiazin-10-yl)propyl)-1-piperazinyl)ethoxy)ethanol, or its pharmaceutically acceptable salts.

6. Therapeutic use according to claim 5 in the treatment or prevention of emesis.

7. A method of preventing or treating, in humans or mammals, disorders or conditions associated with nausea or vomiting, such as radiation induced emesis, cancerotherapy, HIV-therapy, motion sickness, migraine, post operative nausea, Ménière's disease and related disorders, method comprising the administration of 2-(2-(4-((2R)-2-methyl-3-(10H-phenothiazin-10-yl)propyl)-1-piperazinyl)ethoxy)ethanol, or its pharmaceutically acceptable salts to said human.

8. Use of 2-(2-(4-((2R)-2-methyl-3-(10H-phenothiazin-10-yl)propyl)-1-piperazinyl)ethoxy)ethanol, or its pharmaceutically acceptable salts for the manufacture of a medicament for preventing or treating disorders or conditions associated with nausea or vomiting.

9. A process, pharmaceutical composition, use or method according to any of the preceding claims characterized in that the 2-(2-(4-((2R)-2-methyl-3-(10H-phenothiazin-10-yl)propyl)-1-piperazinyl)ethoxy)ethanol is in the form of the dihydrochloride salt.

10. A chemical compound selected from the group consisting of
10-[(2S)-2-methyl-3-(tetrahydro-2H-pyran-2-yloxy)propyl]-10H-phenothiazine;
10-[(2S)-3-chloro-2-methylpropyl]-10H-phenothiazine;
10-[(2R)-2-methyl-3-(tetrahydro-2H-pyran-2-yloxy)propyl]-10H-phenothiazine;
10-[(2R)-3-chloro-2-methylpropyl]-10H-phenothiazine; and
10-[(2)-2-methyl-3-(tetrahydro-2H-pyran-2-yloxy)propyl]-10H-phenothiazine.
